(19) United States
(12) Patent Application Publication
(10) Pub. No.: US 2009/0304670 A1
(43) Pub. Date: Dec. 10, 2009

(54) FOOD PRODUCT COMPRISING A PROLINE SPECIFIC PROTEASE, THE PREPARATION THEREOF AND ITS USE FOR DEGRADING TOXIC OR ALLERGENIC GLUTEN PEPTIDES

(76) Inventors: Luppo Edens, Rotterdam (NL); Emile de Deckere, Vlaardingen (NL)

Correspondence Address: NIXON & VANDERHYE, PC, 901 NORTH GLEBE ROAD, 11TH FLOOR, ARLINGTON, VA, 22203, US

(21) Appl. No.: 12/162,512

(22) PCT Filed: Jan. 30, 2007
(86) PCT No.: PCT/EP07/00896

§ 371 Date: Dec. 19, 2008

(30) Foreign Application Priority Data
Feb. 2, 2006 (EP) 06101219.1

(51) Int. Cl. A61K 38/48; C12N 9/50

(52) U.S. Cl. 424/94.64; 424/94.63; 435/219

(57) ABSTRACT

The present invention relates to a pasteurized food product having a water activity of at least 0.80, preferably at least 0.85 and containing a proline specific protease.

### FIELD OF THE INVENTION

The invention relates to a food product comprising a proline specific protease, the preparation thereof and its use for degrading toxic or allergenic gluten peptides.

### TECHNICAL BACKGROUND

It is known that ingestion of gluten, a common dietary protein present in wheat, barley, rye, spelt and triticale, causes disease in some individuals. Gluten is a complex mixture of glutamine- and proline-rich gliadins and glutenins, which is thought to be responsible for inducing a number of diseases. Due to their amino acid composition, specific parts of these glutens resist proteolytic degradation in the human gastrointestinal tract. As a result, specific, proline-rich peptides can build up and may lead to undesirable effects, such as an intolerance for a variety of such gluten derived peptides. For example, the amino acid sequences of the peptides responsible for the observed toxicity of gluten in patients suffering from celiac disease have been described (Arentz-Hansen et al, J. Exp. Med. 2000; 6:337-342; Vader et al, Gastroenterology 2002; 122:1729-1737).

Celiac disease is a widely prevalent autoimmune disease of the small intestine. Among celiac patients a high prevalence of various autoimmune disorders, especially type I diabetes, dermatitis herpetiformis, autoimmune thyroiditis, collagen diseases, autoimmune alopecia and autoimmune hepatitis, has been observed. Celiac disease is occasionally also accompanied by psychiatric and neurological symptons illustrating the far-reaching consequences a disturbed metabolism of proline-rich peptides may have.

Sofar only a life-long gluten-free diet can effectively prevent the clinical symptoms in celiac disease patients. Unfortunately for these patients, gluten is a cheap protein with interesting application possibilities so that it is applied in a wide variety of food stuffs including commercial soups, soy sauces, sauces, ice creams, potato chips and hot dogs. Gluten intolerant patients thus need detailed lists of foodstuffs to prevent the intake of the problematic gluten molecules. After all, ingestion of gluten quantities as low as 50 mg per day, may induce return of the clinical symptoms.

Nowadays we understand that the problematic, proline-rich peptides present in gluten are highly resistant to cleavage by gastric and pancreatic peptidases such as pepsin, trypsin, chymotrypsin and the like. Only specific enzymes that can hydrolyse peptide bonds involving proline, are capable of extensively hydrolysing proline-rich sequences hereby destroying the epitopes relevant for celiac disease. Various enzymes have been reported to have a beneficial use in the inactivation of toxic proline-rich peptides, such as prolyl oligopeptidases (EC 3.4.21.26; Shan et al. Science 297, p 2275-2279) and dipeptidyl peptidase IV (EC 3.4.14.5; US-A-2002/0041871). Also a number of patent applications have been published mentioning the possible implications of proline specific proteases in lowering the antigenicity of gluten containing food stuffs, as there is WO-A-2002/45523, as well as the use of such enzymes in preventing the clinical symptoms of celiac and related diseases as, for example, WO 03068170 and WO 2005/027953. Very recently the validity of the enzyme therapy in treating celiac patients was demonstrated by using duodenal extracts (Cornell et al., Scandinavian Journal of Gastroenterology, 2005; 40: 1304-1312).

WO-A-2002/45523 specifies a proline specific endoprotease for use in the proteolysis of polypeptides, including proline-rich peptides. It describes the incorporation of the endoprotease in proteinaceous food products to suppress bitterness or to reduce their allergenicity. It is recognised in WO-A-2005/027953 that this particular endoprotease is ideally suitable as a dietary supplement supporting the digestion process of dietary gluten, as it exhibits a broad pH optimum that allows the enzyme to be active in the mouth, the esophagus, the stomach and to continue its activity in the duodenum.

WO-A-2005/027953 aims at the removal of toxic proline-rich peptides from food prior to consumption, thus preventing or minimising exposure of the body to toxic proline-rich peptides. It also teaches the use of stabilised enzyme formulations as a digestive aid. In this approach the enzyme is consumed together with the foodstuff in order to degrade the proline-rich and/or glutamine-rich protein sequences of the foodstuff during passage of the gastrointestinal tract. However, according to WO-A-2005/027953, the enzyme formulation may only be incorporated in proline-rich and/or glutamine-rich foodstuffs having a water activity below 0.85 to keep the enzymes sufficiently active. In case the food product is to be stored for longer periods, contact with moisture or humid air should be avoided, thus suggesting the use of dried foods. This assumption puts restraints on the number of choices of foodstuffs the endoprotease can be combined with.

The use of enzyme formulations in water-containing foodstuffs such as margarine or similar spreads in general is widely known in the art. However, in most cases the enzymes are added as processing aids and a prolonged enzymatic activity, i.e. an activity that continues after packaging of the product, is not intended. For instance, DE-A-101 04 945 teaches low-fat spreads containing phospholipids and enzymes, for instance transglutaminase, without the use of emulsifiers or stabilisation aids. After a short incubation period during the production phase of the product, the transglutaminase is inactivated by heating at 95° C. for 2-3 minutes.

WO-A-95/28092 concerns the use of stabilisation aids, such as polyols, to stabilize water-in-oil emulsions suitable for foods, wherein the emulsions comprise a heat-labile compound, such as an enzyme. Contrary to the two above mentioned applications, WO-A-95/28092 aims at a long term stabilisation of enzymatic activities. To that end, amounts of 40 and 50% glycerol in the water phase are exemplified. However, the incorporation of such high amounts of polyols in food products is either not allowed or is organoleptically unacceptable.

### DESCRIPTION OF THE INVENTION

It is now found that proline specific proteases may be used as an ingredient in food products exhibiting high water activities and lacking high amounts of enzyme stabilisers. Such products may even be pasteurized to guarantee adequate shelf stabilities without dramatic losses of the relevant enzyme activity. In the present application it is demonstrated that in food products such as sandwich fillings, toppings, condiments, sauces, various beverages and emulsions such as low fat spreads, the proline specific proteases remain sufficiently active to achieve adequate gastric hydrolysis of proline-rich gluten sequences. In general the taste of the food product is not affected or altered by the presence of the enzyme.

The fact that the enzyme survives the pasteurization treatment is surprising, since it is believed in the art that under conditions of a high water activity the vast majority of enzymes cannot survive pasteurization conditions. Similarly the vast majority of enzymes is expected to become inactive within a week if stored in products having a high water activity. Therefore, it is also surprising that according to the invention the enzyme maintains its activity during periods up to one year if the food product having a high water activity is stored under refrigerated conditions. Under refrigerated conditions is meant temperatures of below 10° C., preferably between 0 and 10° C., more preferably between 2 and 8° C.

The invention thus relates to pasteurized and shelf stable food products having a water activity of at least 0.80, preferably of at least 0.85 and containing a proline specific proteolytic activity which is high enough to detoxify proline-rich protein sequences. Toxic quantities of proline rich protein sequences are considered to be present in gluten quantities higher than 1 mg.

According to an other aspect of the invention a shelf stable food product is disclosed having a water activity of at least 0.80 preferably at least 0.85 and containing a proline specific proteolytic activity which is capable to detoxify proline-rich protein sequences, whereby the food product comprises less then 1 w/w % of protein or peptides and preferably the food product is a gluten fee.

The present invention also relates to sterile proline specific protease. With sterile is meant free of microorganisms, preferably also free of microbial spores. The proline specific protease is preferably filtered free of microorganisms, preferably also free from microbial spores.

Cereal proteins can be subdivided into albumins, globulins, prolamins and glutelins. Gluten is the water-insoluble protein fraction of cereals like wheat, rye, spelt, oats, barley, maize and rice that remains after washing to remove starch and water-soluble components. It can be subdivided into gliadins and glutenins. The glutenins can be subdivided into high and low molecular weight subunits. For a further discussion of gluten proteins, see Wheat Gluten (P. R. Shewry and A. S. Tatham eds., Cambridge: Royal Society of Chemistry, 2000) or the review by Wieser (1996) Acta Paediatr. Suppl. 412:3-9.

According to the internationally recognised schemes for the classification and nomenclature of all enzymes from IUMB, oligopeptidases, dipeptidylpeptidases and endoproteases are those enzymes that hydrolyse internal peptide bonds, which are then divided in sub-subclasses on the basis of their catalytic mechanism. The preferred proline specific protease suitable for the purpose of the invention is the acid-stable and pepsin-stable endoprotease from A. niger as disclosed in WO-A-02/45524 and WO-A-2005/027953, which is able to cleave peptides and intact proteins at the carboxyl side of proline residues and which also is able to cleave peptides and intact proteins under very low pH conditions and in the presence of pepsin. This endoprotease survives the presence of the enzyme pepsin under acid conditions and is likely to continue its activity throughout the duodenum. The most preferred endoprotease is a proline specific endoprotease derived from the food grade fungus Aspergillus or a proline specific endoprotease belonging to the S28 family of serine proteases.

Water activity is the relative availability of water in a substance. It is defined in the art as the vapour pressure of water divided by that of pure water at the same temperature. Therefore, pure distilled water has a water activity of exactly one. Water activity is different from moisture content (% water) in a food product. Moisture content is the total moisture, that is, the amount of bound plus free water present in the sample, whereas water activity only provides a measurement of the free moisture and is usually expressed as a_{w} or percentage Equilibrium Relative Humidity (% ERH). The water activity of a food product is the constant relative humidity of the air in direct vicinity of the food product when equilibrium between the food product and the surrounding air is established. This constant relative humidity is then called ‘% ERH’ if it is expressed on percentages (0 to 100%), or ‘water activity’ if it is expressed as values between 0 and 1.0. Methods for water activity determinations are detailed in the official methods of analysis of AOAC International (1995), Method 978.18.

By heat treatment in the present specification is meant a heat treatment of at least 65° C., preferably at least 70° C., and for at least 2 seconds, preferably for at least 20 seconds. An example of such a heat treatment is a pasteurization as applied for milk i.e. heating at 72° C. for 15 seconds. Pasteurisation is a concept known to the skilled person. The resulting food product is thus a microbially safe product having an improved shelf life.

By a food product is meant a product or a food ingredient which is intended for consumption without prior heat treatments such as baking, frying or cooking.

A food product having an extended or improved shelf life is understood as having a shelf life of at least one week up to a year or more, during which the organoleptical properties as well as the microbial safety of the product are guaranteed. Obviously the allowable shelf life strongly depends on the actual storage conditions of the food product. Many perishable food products have to be stored cool in order to maximize their shelf lifes.

If stabilisation aids are used in the food product of the invention, in particular polyols such as glycerol, sorbitol, sucrose, polypropylene glycol, trehalose, maltodextrins, lactose and glucose, the amount thereof is in general less than 10 wt %, preferably less than 5 wt % of the food product.

Preferably the food product according to the invention contains less than 1% w/w casein, more preferably the food product according to the invention contains no casein.

The intake of the proline specific proteases in the form of a pill or a tablet might allow a gluten-intolerant patient to consume such gluten-containing food products safely. However, it is now found that the protease may also conveniently be incorporated into food products which, in itself, may contain no gluten or low amounts of gluten, but which food products are commonly combined with gluten-containing foodstuffs. More preferably, the food products according to the invention containing endoprotease are food ingredients that are considered as “gluten-free” in the art. According to the “Codex Standard for Gluten-Free Foods” (Codex Stan 118-198) of the Codex Alimentarius, the nitrogen content of food ingredients derived from gluten-containing cereals may not exceed 0.05 g (50 mg) per 100 g product on a dry basis, when they are used in a gluten-free food.

According to the present invention a food product is disclosed which comprises a proline specific proteolytic activity of higher than 0.5 PPU per serving i.e. the enzymatic activity present in one serving can hydrolyse 25 mg of gluten. One serving is the amount of food consumed during one meal so in general within one hour, preferably within 40 minutes.

Food products preferred as a carrier for proline specific proteases are those food products that are stored refrigerated. It is especially preferred that the protease containing food product is a condiment, i.e. a foodstuff that is used to enhance the flavour of other foods, especially gluten-containing foods. Condiments have the advantage of being abundantly present at home and in restaurants, diners and supermarkets, and typically have of a prolonged shelf life. Preferred examples of condiments are tomato sauce or tomato ketchup. Such products typically have a pH below 4.2, more preferably below 4.0 which implies that they require a limited pasteurisation treatment only. Examples of other acid products requiring limited pasteurisation treatments and which are perfectly suitable as carriers for an active proline specific protease are fruit juices and fruit concentrates. In fact even acidified or carbonated bottled water would present an excellent carrier for the enzyme. “Shots” like vegetable or fruit concentrates also fall within this category. Likewise acid products containing a food grade preservative like benzoate or sorbate present excellent carriers for the enzyme. For example, toppings or sandwich fillings typically consumed in combination with gluten containing food such as bread, having water activity values above 0.85. Also very acid products that require no pasteurisation at all, such as cola's, present an excellent carrier as the proline specific protease is remarkably stable under these conditions. Furthermore, the enzyme is quite compatible with preparations containing high concentrations of viable probiotics. Usually such probiotic products have a water activity higher than 0.95 and are stabilized by a pH below 4.0.

Additionally the invention relates to a process for the preparation of a food product containing the enzyme formulation of the invention, wherein a proline specific protease is added to the food product after the food product was subjected to a pasteurisation treatment. In this approach the enzyme can be added sterile to an already pasteurized product. An example of an enabling technology for such an approach is the aseptic dosing technology as is for example sold by TetraPak (Tetra Aldose™ S; see e.g. http://www.tetrapak-processing.de/produkte/pdf/aldose.pdf).

Yet another embodiment of the invention is a water-in-oil or an oil-in-water emulsion, a spread, preferably a margarine or a low fat spread. The widely used low fat spreads intended for consumption together with gluten containing foodstuff are exceptionally suitable as a carrier for the enzymatic digestive aid. The high water content of these products allows the incorporation of large amounts of enzyme and the product is typically stored at cool conditions, conventionally at temperatures of 7° C. or lower. Because the enzyme is confined to the water phase, such emulsions also fall in the category of products having a water activity of at least 0.85.

Other important advantages of this embodiment of the invention is that the bread and spread are thoroughly mixed in the mouth hereby initiating the degradation of the gluten molecules by the proline specific protease. Furthermore, the presence of fatty compounds is known to inhibit gastric emptying via an orosensory mechanism. Both mechanisms result in more intense and longer interaction periods between enzyme and gluten so that enzyme and gluten can maximally interact before the chyme reaches the duodenum. This is important as the duodenum is known to be the most upstream part of the gastrointestinal tract that can provoke the pathogenic reactions of proline rich gluten molecules. It was found that the proline specific protease, in particular the acid-stable proline specific endoprotease from A. niger according to WO-A-2002/45523, has a broad pH optimum which allows it to be active in the mouth, the esophagus, the stomach and in the duodenum. The fact that proline specific endoprotease exhibits such high residual activities if incorporated into an emulsion, is quite unexpected. The existing literature is rather unanimous in their conclusion that the contact with emulsifiers and the subsequent incorporation into emulsions exerts a significant stress upon enzymes and easily leads to enzyme inactivation (see for example Gatorae et al in “Stability and Stabilisation of Enzymes, Elsevier Sci. Publish. 1993, p 329 or De Roos and Walstra, Colloids and Interfaces B; Biointerfaces 6 (1996) 201-208). Thus especially suited for the present invention is an enzyme:
having proline-specific endoprotease activity and
having an amino-acid sequence identical to SEQ ID NO:2 of WO 2002/45523 or having an amino acid sequence which has at least 80%, preferably at least 90% amino acid sequence identity with amino acids 1 to 526 of SEQ ID NO:2 of WO 2002/45523. The level of identity between amino acid sequences is determined by the method mentioned in WO 2002/45523 page 15.

The enzyme formulation according to the invention has to be incorporated into the food product in an amount that corresponds with the total amount of protein to be digested. For example, a low fat spread is typically applied on a slice of bread. Per slice of bread of 18 grams typically 5 grams of spread is applied. Bread typically contains 8% of protein of which 7% is gluten, i.e. one slice of bread contains 1.5 grams of protein. To achieve adequate hydrolysis during gastrointestinal digestion of all proteins present, i.e. including the gluten fraction and using the enzyme described in WO 2002/45523, approximately 20 PPU per gram of protein present is required (see Materials & Methods section for definitions). Therefore, digestion of 1.5 grams of protein requires 1.5 times 20 PPU corresponding with 30 PPU. These 30 PPU have to be provided by the 5 grams of low fat spread which implies an enzyme activity of 6000 PPU/kg of the low fat spread. The incorporation of the enzyme formulation according to the invention hardly affects the preparation and properties of a low fat spread. However, to avoid the effect of bitterness which is often reported in milk products upon treatment with enzymes having proteolytic activity, the spread according to the invention is preferably devoid of hydrophobic proteins such as caseins. Thus, the taste of the spread may be improved by incorporating a fermented whey protein to provide the typical buttery flavor. We have found that the residual proteins as present in the fermented whey are hydrolysed by the proline specific enzyme but this has no negative organoleptic effects. The proline specific protease is preferably added to the water phase prior to forming the emulsion, most preferably the proline specific protease is added sterile after pasteurisation of the water phase but prior to forming the emulsion. Several methods exist for an efficient production of low fat spreads. According to one method, the oil phase, containing emulsifiers, flavors, vitamins and colors is kept moderately heated and mildly agitated in order not to affect adversely the oil quality. Aqueous phase and oil phase are then mixed and fed into the votator. More detailed descriptions of the preparation of emulsions can be found in the literature a. o. Moustafa in: Practical handbook of Soybean Processing and Utilization; David R. Erickson editor; a joint publication by AOCS Press and United Soybean Board. Nowadays spreads incorporating health promoting compounds such as phytosterols/stanols or arachidonic acids are enjoying increased popularity. Because these are oil-soluble compounds, the stability of the enzyme in the water phase is not affected by such compounds.

Thus the invention relates to a process for the preparation of a food product containing the enzyme formulation of the invention, wherein a proline specific protease is added to the food product either before or after subjecting the food product to pasteurisation.

In principle, other enzymes that are capable of hydrolysing peptide bonds involving proline residues, like prolyl oligopeptidase (EC 3.4.21.26) or dipeptidyl peptidase IV (DPP IV, EC 3.4.14.5) or dipeptidyl peptidase 11 (DPP II, EC 3.4.14.2) could also be applied. These proteases have been listed by a. o. Augustyns et al (Current Medicinal Chemistry, 2005, 12, 971-998) However, in order for a proline specific protease to be suitable as a digestive aid in a food product, either a gluten-containing food or a food product intended for use in combination with gluten-containing foods, the proline specific protease should: i) exhibit pH optima that allows adequate activity under the acid pH values prevailing in the stomach, preferably below pH 5; ii) survive the presence of the gastrointestinal proteolytic enzyme pepsine under these acidic conditions; iii) remain active in a water-containing environment for at least the shelf-life of the food product. Furthermore it is possible to include a co-enzyme to obtain a synergy towards the hydrolysis of gluten proteins. Obviously, the co-enzyme has to satisfy the same stability requirements as the proline specific enzyme in the food product. The invention also relates to the use of proline specific protease for use as a digestive aid in the manufacture of a pasteurized food product having a water activity of at least 0.85 as described above, for prevention of the clinical symptoms of celiac disease or disorders related therewith. Preferably, the food product is intended for consumption in combination with a gluten-containing food product.

Celiac disease is caused by an intolerance to certain proline- and glutamine rich peptides. Incomplete degradation of these peptides contributes to the development and the severity of celiac disease. Celiac disease is occasionally accompanied by psychiatric and neurological symptoms. Already in 1979 Panksepp (Trends in Neuroscience 1979; 2:174-177) proposed the opioid excess theory in which he suggested that a disturbed opioid metabolism is part of the pathogenesis in autism. Therefore, a food product containing the endoprotease of the invention can also be used by patients suffering from psychiatric disorders including autism, schizophrenia, ADHD, bipolar mood disorders and depression, which are all linked with the consumption of proline-rich dietary proteins. Other disorders related with celiac disease comprise autoimmune disorders, especially type I diabetes, dermatitis herpetiformis, intestinal cancers, intestinal non-Hodgkin's lymphomas, autoimmune thyroiditis, collagen diseases, auto-immune alopecia and autoimmune hepatitis. Furthermore the Irritable Bowel Syndrome (IBS) has been linked with the hard-to-digest proline-rich protein sequences. Patients that can benefit from the present invention may suffer from any of these aforementioned disorders. Such patients may be of any age and include adults and children. Children in particular benefit from prophylactic benefits, as prevention of early exposure to toxic gluten peptides can prevent initial development of the disease. The incorporation of proline specific endoprotease formulation is especially advantageous for this category of patients, because of the popularity of condiments, particularly of mayonnaise and ketchup, to this group. Children eligible for prophylaxis can be identified by genetic testing for predisposition, e.g. by HLA typing, by family history, by T cell assay, or by other medical means.

### LEGENDS TO THE FIGURES

FIG. 1: Levels of T-cell stimulating epitopes recovered from the “stomach” without the proline specific endoprotease. Conditions were as explained in Example 3. “Alpha” refers to the level of reactive alpha-gliadin molecules, “gamma” to the level of reactive gamma-gliadin molecules, “HMW” to the level of reactive HMW-glutenins and “LMW” to the level of reactive LMW-glutenins.

FIG. 2: Levels of T-cell stimulating epitopes recovered from the “stomach” containing the proline specific endoprotease. Conditions were as explained in Example 3. See legend of FIG. 1 for the explanation of “alpha”, “gamma”, “HMW” and “LMW”.

FIG. 3: Levels of T-cell stimulating epitopes pellets recovered from the “stomach” with and without proline specific endoprotease added and tested in a Western blot treated with anti alpha-gliadin. Conditions were as explained in Example 3.

FIG. 4: Percentage of residual enzyme activity in the water phase after melting a low fat spread at 53° C., adding the proline specific endoprotease to the water phase and shaking the water/fat mixture for 10, 70 and 100 minutes at 53° C. Conditions were as explained in Example 4.

FIG. 5: Shelf stability of the proline specific endoprotease in the aqueous phase of a low fat spread kept at various temperatures. Conditions were as explained in Example 5.

### MATERIALS & METHODS

### Enzyme Activity Tests

The A. niger proline specific endoprotease activity was tested using CBZ-Gly-Pro-pNA (Bachem, Bubendorf, Switserland) as a substrate at 37° C. in a citrate/disodium phosphate buffer pH 4.6. The reaction products were monitored spectrophotometrically at 405 nM. The increase in absorbance at 405 nm in time is a measure for enzyme activity. A Proline Protease Unit (PPU) is defined as the quantity of enzyme that releases 1 μmol of p-nitroanilide per minute under the conditions specified and at a substrate concentration of 0.37 mM Z-Gly-Pro-pNA.

### Quantitative Detection of T Cell Stimulatory Epitopes

The concentration of T cell stimulatory epitopes of both gliadin and glutenins in the soluble fractions of the dynamic gastrointestinal in vitro model was determined using monoclonal antibody based (mAB) competition assays. To that end the samples were diluted in a buffer containing 50 mM Na₂HPO₄/NaH₂PO₄ pH 7.0, 150 mM NaCl, 0.1% Tween-20 and a protease inhibitor cocktail (Complete, Roche Diagnostics GmbH, Penzberg, Germany). The assays were performed in duplo as described previously (Spaenij-Dekking et al, (2004)Gut 53, 1267-1273).

### Protein Analysis by 1 D SDS-PAGE and Western Blotting

To determine the level of T cell stimulatory epitopes present in the solid (precipitated) fractions of the different samples of the dynamic gastrointestinal in vitro model, 1 D SDS-PAGE experiments were performed. The solid fractions were solubilized in 6× protein sample buffer (60% glycerol, 300 mM Tris (pH 6.8), 12 mM EDTA pH 8.0, 12% SDS, 864 mM 2-mercaptoethanol, 0.05% bromophenol blue) and run on a 12.5% SDS-PAGE gel. The proteins were visualized either directly using Imperial Protein Stain (Pierce, Rockford Ill., USA), or after Western blot to PVDF membranes with the mAbs specific for stimulatory T-cell epitopes from α- and γ-gliadin (Spaenij-Dekking et al, (2004) Gut 53, 1267-1273) and HMW and LMW-glutenins (Spaenij-Dekking et al., Gastroenterology 2005; 797-806).

### Antibody Based Competition Assay

For the generation of an antibody-based assay, monoclonal antibodies were raised in Balb C mice against known T cell stimulatory alpha-, gamma-gliadin and a LMW glutenin peptide. After fusion of the spleens of the mice with a mouse myeloma cell line, antibody-producing hybridomas were obtained. These were cloned by limiting dilution and the mAbs secreted by these cells were tested for their use in a mAb competition assay. For each of the specificities one or two suitable mAb were selected and the epitopes recognized by the different mAb were determined (Table 1).

TABLE 1

| specificity | T cell epitope | Ab epitope |
|---|---|---|
| α-gliadin (α9) | QLQPFPQPQLPY | QPFPQPQ |
| (α20) | PFRPQQPYPQPQPQ | RPQQPYP |
| γ-gliadin | QPQQPQQSPFQQQRF | QQRPFI |
| LMW glutenin | QPPFSQQQQSPFSQ | QSPFS or PPFSQQ |
| HMW-glutenin | GYYPTSPQQS | |

With the mAbs competition assays were developed by which T cell stimulatory epitopes present in both intact proteins and small peptides with sizes of about 11 amino acids (the size of a T cell epitope) can be detected quantitatively at low levels.

In a competition assay different dilutions of the samples are mixed with a fixed concentration of a biotinylated indicator peptide (which encodes the T cell epitope). For quantification of the gliadin assays a standard curve was made using the European gliadin reference IRMM-480 in a concentration range of 10 μg/ml-10 ng/ml. The assay for LMW glutenin was quantified using a synthetic peptide encoding the T cell stimulatory epitope in a range from 1 μg/ml-1 ng/ml. Whereas the assays for HMW glutenin were quantified using a pepsin/trypsin digest of recombinant HMW glutenin proteins in a range from 1 μg/ml-1 ng/ml. The presence of antibody bound biotinylated peptide was visualized with labeled streptavidin

### Example 1

### Filter Sterilization of the Proline Specific Endoprotease

Filtration presents a preferred option for sterilizing enzymes. The enzyme solution to be sterilized may be obtained after chromatographic purification and the enzyme solution may comprise one or more solvents or other additives to adjust the enzyme activity and to further stabilize the enzyme. Suitable stabilizers are, for example, sorbitol and glycerol. Glycerol solvents may be added to a concentration of from 10 to 70 w/w %, or more preferably 30 to 60% w/w, of the enzyme solution.

Filter sterilization can be accomplished by pumping the enzyme solution through a sterile filter. Preferably the filter sterilization is carried out by a prefiltration followed by a second filtration through a 0.22 nm cartridge filter. The thus sterilized enzyme solution can be added via a sterile dosing device into a holding vessel containing a previously pasteurised or sterilized aqueous food product or food ingredient. The enzyme containing product or food ingredient can be directly packed. If the enzyme solution has to be incorporated into a fat spread or a low fat spread, the sterilized enzyme solution can be mixed with the pasteurized aqueous phase which is then emulsified with a fat or oil at the appropriate, elevated temperature.

To obtain a sterilized enzyme solution for use on a laboratory scale, a solution of the proline specific endoprotease as obtained from A. niger was filter sterilized by the following procedure. A syringe was filled with 1 ml enzyme concentrate, and a sterile filter, Millex GV 0.22 μm from Millipore with a surface of 4.91 cm², was placed on top of the syringe. Upon applying hand pressure the enzyme solution was pushed through the 0.22 μm Millipore filter hereby providing a sterilized solution with a enzyme activity corresponding with the activity of the enzyme solution prior to the sterile filtration.

### Example 2

### The Digestion of Bread in a Dynamic GastroIntestinal In Vitro Model in the Absence And in the Presence of a Proline Specific Endoprotease

The passage of food through the gastrointestinal tract is a very dynamic process which cannot be simulated in static in vitro models. The dynamic gastrointestinal in vitro model as developed by TNO (Zeist, The Netherlands) is a validated digestion model that simulates in high degree the successive dynamic processes in the stomach and in the small intestine (Minekus et al, ATLA 1995, 23, 197-209; Larsson et al, J Sci Food Agic 1997, 74, 99-106). Results obtained in these models have shown very good resemblance with the results obtained in studies with humans and animals.

To test the digestion of white bread in the absence and in the presence of the A. niger derived proline specific endoprotease, an experiment was carried out in this dynamic gastrointestinal model. These experiments were performed under the average physiological conditions of the gastrointestinal tract as described for young adults after the intake of a semi-solid food. The bread was first “chewed”, i.e. carefully mixed with salivary enzymes during 5 minutes and in the absence (reference) or the presence of the proline specific endoprotease from A. niger. In the actual experiment 70 grams of white bread (containing 5 gram of gluten) was homogenized together with 11 ml of gastric juice in the absence or presence of 100 PPU proline specific endoprotease. After homogenization 25% of the mixture was added to the in vitro digestion system. In the gastric compartment the pH was gradually decreased by the secretion of gastric acid. The ‘swallowed’ salivary enzymes (amylase) was immediately present whereas the gastric enzymes (pepsin and gastric lipase) were gradually secreted. The pepsin became active at pH below 5.0. During 2.5 hours the gastric contents were gradually delivered into the small intestine via the ‘pyloric valve’. In the duodenum compartment the pH was controlled at pH 6.5 by the secretion of bicarbonate. Pancreatic juice containing amylase, lipase and proteolytic enzymes (e.g. trypsine and chymotrypsine) and bile were gradually secreted into the duodenal compartment. The secretion products were mixed through the food coming from the stomach by peristaltic mixing and gradually transferred to the jejunal and ileal compartments.
After 4-5 hours approximately 80% of the small-intestinal contents was gradually delivered into the ‘large intestine’ (sampling bottle) via the ‘ileo-caecal valve’. Digested compounds were dialysed continuously from the jejunal and ileal compartments of the model via semi-permeable hollow fibre membrane systems. The dialysis bottles were changed every 2 hours.

During the passage of the gluten proteins (and the food) through the compartments of the dynamic system, small samples of approximately 2 ml were taken from the gastric, duodenal and jejunal compartments at the following time points: t=0, 15, 30, 45, 60, 90, 120, 150, 180 and 240 min. Immediately after collection, the samples were frozen in dry ice in order to stop the enzyme activity. At the end of each experiment the residues in the various compartments were collected on dry ice and stored in two tubes of 10 ml each at minus 20° C.

### Example 3

### Testing of In Vitro Digested Bread for the Presence of Toxic Gluten Epitopes

Two types of reagents are available that can be used to measure the presence of gluten peptides in food samples: T cell clones that have been isolated from the small intestine of celiac disease patients and monoclonal antibodies specific for various gluten peptides. The T cell clones respond to gluten peptides when these are bound to the disease predisposing HLA-DQ2 or HLA-DQ8 molecules. These inflammatory T cell responses are believed to be the primary cause of celiac disease. T cell clones specific for peptides in alpha, gamma-gliadin, LMW-glutenin and HMW-glutenin are available (c.f. Wal van de, Y. et al., Eur. J. Immunol. 29, 3133-3139 (1999) and Vader et al., Gastroenterology, 122: 1729-1737 (2002). Monoclonal antibodies specific for T cell stimulatory alpha-gliadin, gamma-gliadin and Low Molecular Weight (LMW) and High Molecular Weight (HMW)-glutenin peptides are also available and have been incorporated in a competition assay for the detection of these peptides in food samples (Spaenij-Dekking et al., Gut, 53: 1267-1273 (2004)).

Stomach and duodenum fractions collected at time points t=0, 15, 30, 45, 60, 90 and 120 minutes, prepared with and without adding the proline specific endoprotease were pre-treated according to the following protocol intended to inactivate the proline specific endoprotease. First the pH of samples was raised to 11-12 using 1 M NaOH and then immediately neutralized using 1 M HCL. After a centrifugation for 10 minutes at 14.000 rpm, the supernatants as well as the pellets of the various samples were collected and heated for 10 minutes at 85° C. to stop any remaining enzymatic activities. Of the supernatants, dilutions were prepared of 1:40; 1:200; 1:1000 and 1:5000 in 20 mM phosphate buffer pH7, 150 mM NaCl, 0, 1% Tween-20, 2× protease inhibitor mix without EDTA. These dilutions as well as the pellet fractions were stored at −20° C. until measurement next day.

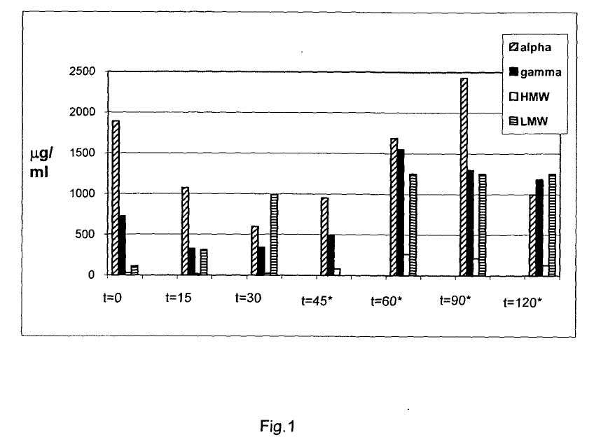

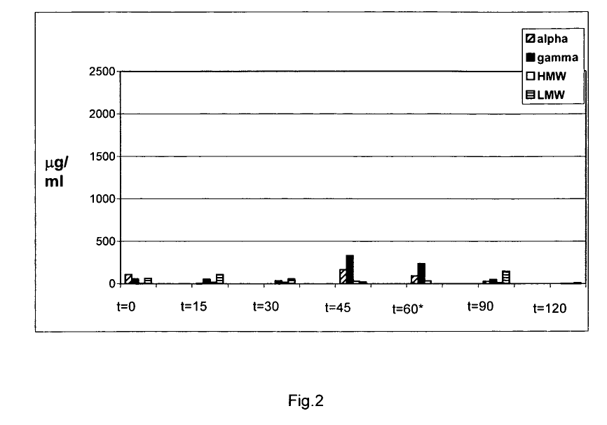

After thawing the samples, the water-soluble fractions of the samples were tested in the competition assay with monoclonal antibodies specific for alpha- and gamma-gliadin and for LMW- and HMW-glutenins. For this purpose the samples were treated as indicated in the Materials and Methods section and several dilutions were measured. The results obtained with these water soluble stomach samples are shown in FIG. 1 (generated in the absence of proline specific endoprotease) and in FIG. 2 (generated in the presence of the proline specific endoprotease). With the exception of HMW-glutenins, all gluten components could be detected. The data obtained clearly show that the addition of the proline specific endoprotease from A. niger has a dramatic effect on the presence of these gluten proteins in the soluble fraction: even at t=0 (i.e. less than a minute after adding the enzyme to the oral preparation) a strong decrease of the presence of gluten proteins/peptides can be observed.

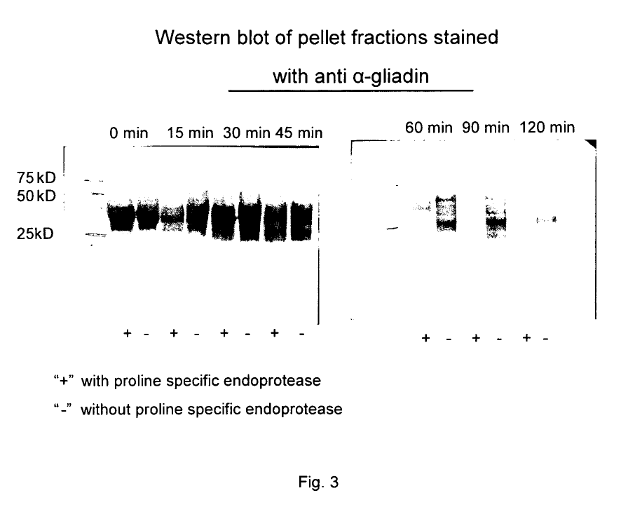

In a separate experiment the water-insoluble fractions (i.e. the pellet) of the stomach samples were subjected to SDS-PAGE followed by transfer of the separated proteins onto PVDF-membrane. These membranes were then stained with the alpha-gliadin specific antibody (FIG. 3). While the antibody detected gliadin in all fractions without the proline specific endoprotease, the addition of the proline specific endoprotease led to a strong reduction in the signal after the 45 minutes timepoint. At time points 90 and 120 minutes hardly any gliadin could be detected in the stomach samples obtained from material digested in the presence of the proline specific endoprotease.

From these data it can be concluded that the proline specific endoprotease is highly efficient in breaking down gluten molecules once these are in the water-soluble fraction. As the antibodies used in this assay are specific for amino acid stretches that are shorter as those required for T cell stimulation, this indicates that the treatment with the proline specific endoprotease results in a strong reduction of potentially harmful gluten-like molecules in the water-soluble fraction. Because especially these water soluble peptides can be expected to efficiently interact with the receptor sites relevant for celiac disease, these data obtained with the water soluble gluten fraction are highly relevant for in vivo conditions.

Quite surprisingly, the proline specific endoprotease also is capable of hydrolysing gluten molecules that are present the water-insoluble phase. After 90 minutes gliadin molecules could not longer be detected in the fractions that were treated with the enzyme while such molecules were still present in the control samples.

Together the results described in this Example indicate that the proline specific endoprotease according to the invention is capable of degrading gluten under conditions that mimic the conditions present in the human stomach. Moreover, the enzyme can do that so efficiently that virtually no toxic gluten epitopes remain.

### Example 4

### Compatibility of the Proline Specific Endoprotease with the Production of a Low Fat Spread

To test whether or not the activity of the proline specific endoprotease would survive an exposure to emulsifiers at elevated temperatures and an incorporation in a water/oil emulsion, the following test was carried out.

In a local supermarket a low fat spread “Halvarine voor op brood” (40% fat) as produced by Winner Food BV, Lopik, The Netherlands, was purchased. Listed ingredients are mono- and diglycerides of fatty acids (E471) as emulsifier, sorbic acid (E200) as preservative, citric acid, vegetable oils and fats, water, flavours, vitamins A and D and salt. The melting behavior of the low fat spread was tested in a shaking, thermostated incubator. At 53° C. the low fat spread was completely liquefied resulting in a slow separation of aqueous and fat layer.

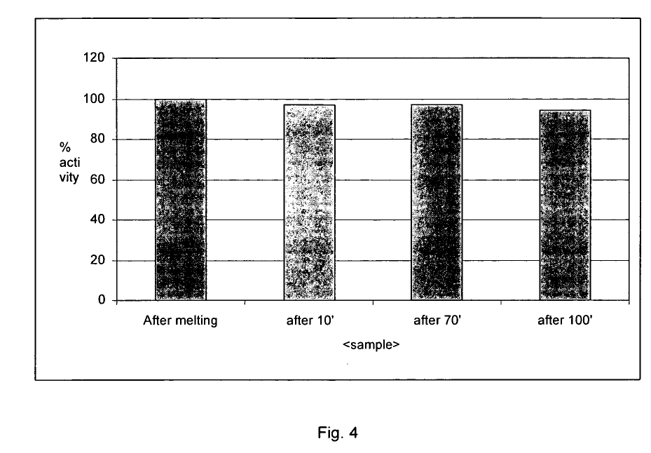

To enable the incorporation of an adequate amount of the proline specific endoprotease into the low fat spread, 15 ml of a liquid enzyme concentrate containing approximately 10 PPU/ml (see Materials & Methods for the enzyme definition) was freeze-dried in a 50 ml Greiner tube. The dried powder (about 2.25 grams) was mixed with 25 grams of the low fat spread, after which the mixture was melted at 53° C. in a shaking thermostated incubator hereby dissolving the freeze-dried enzyme powder. Immediately after the melting and spontaneous separation of water and fat, a sample (300 μl) was withdrawn from the water layer in the tube and cooled to room temperature. After withdrawal of this first sample, the tube containing the liquefied emulsion with the enzyme was vigorously shaken for 10 seconds and left at 53° C. After 10, 70 and 100 minutes again 300 μl samples were withdrawn from the aqueous layer followed by vigorous shaking after sample withdrawal. Finally all samples obtained from the aqueous layer were diluted 1000 times with 100 mM acetate buffer pH 4.2 and residual enzyme activity was measured in a microtiter plate (MTP) assay. To that end, hydrolysis of the synthetic peptide substrate Ala-Ala-Pro-pNA (Pepscan, Lelystad, The Netherlands) yielding the tripeptide Ala-Ala-Pro and the coloured pNA molecule, was followed at 405 nm using 10 minutes kinetic measurements at 40° C. in a TECAN Genios MTP Reader (Salzburg, Vienna). Substrate Ala-Ala-Pro-pNA (rather than Z-Gly-Pro-pNA) was used because Ala-Ala-Pro-pNA allows the measurement of much smaller enzyme quantities. Each well contained 250 μl substrate solution, 3 mM AAP-pNA in 100 mM acetate buffer pH 4.2 and was pre-heated in a Tecan Genios MTP reader for 10 minutes at 40° C. The reaction was started by adding 50 μl of an appropriate enzyme dilution (in this case 1000 times). Liberation of the pNA molecule was followed for 15 minutes. Data collection was carried out with Magellan software (Tecan). The increase in optical density at 405 nm was recorded and further processed in Excel to yield the picture shown in FIG. 4. The activity of the proline specific endoprotease immediately after melting the spread was defined to be 100%. As shown by the results, the enzyme activity is hardly affected by the low fat spread environment at 53° C. Even shaking the melted spread to mimic the emulsifying process had little or no influence notwithstanding the resulting excessive foaming.

### Example 5

### Shelf Stability of the Proline Specific Endoprotease in the Aqueous Phase of a Low Fat Spread

To test the shelf stability of the proline specific endoprotease in the water phase of a low fat spread, a lactic acid containing waterphase having a pH of 4.5 and a wateractivity of 0.98 was prepared. To prevent microbial contamination of the water phase, a concentrated solution of sodium benzoate was sterile added to reach a concentration of 600 ppm. Then sterile filtered proline specific endoprotease was added sterile to reach an enzyme activity of 15 PPU/gram. This solution was then divided over a large number of small, sterile vials. Some of these vials were placed at minus 20° C. to serve as a reference, other vials were placed at 8° C. and at 30° C. Every few weeks the remaining proline specific enzyme activity was measured in the various vials. Enzyme activities were measured according to the procedure detailed in the Materials & Methods section.

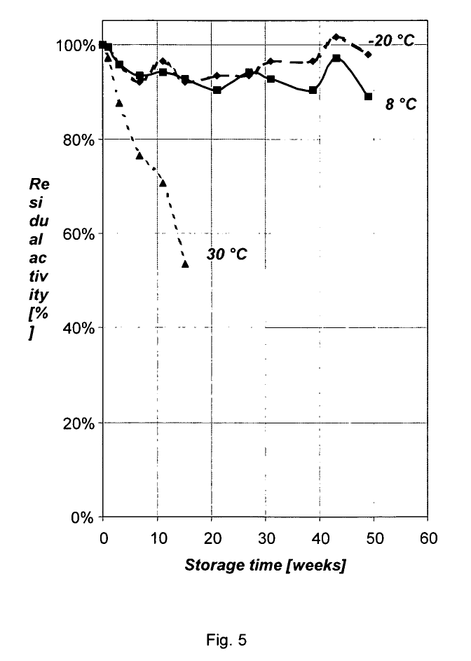

The results, shown in FIG. 5, illustrate that if kept at an ambient temperature of 8° C., the enzyme remains perfectly active for a period of at least 50 weeks under these high a_{w} conditions.

### Example 6

### In Vitro Digestion of Toasts with an Enzyme Containing Raspberry Topping

To illustrate the concept of using an enzyme containing topping for facilitating the breakdown of toxic gluten molecules in food, the following experiment was carried out. First 250 ml of a shelf stable raspberry topping was prepared. To a quantity of 204 g of liquid enzyme concentrate (12 PPU/ml), 20.7 grams of sucrose, 1.0 grams of citric acid, 0.02% of a raspberry flavour (Givaudan, 76525-36) and 20.7 g of a 0.5% solution of saccharine in water was added. After dissolution of the various ingredients, 3, 11 gram xanthan (Keltrol RD, CP Kelco, II) was added and dissolved by careful stirring. The final pH of the viscous mass was 3.7, the final enzyme concentration about 9.8 PPU/g topping, the final water activity about 0.99. The benzoate concentration was adjusted to about 600 ppm. In the same way a placebo product containing all ingredients mentioned but without the active, proline-specific enzyme, was prepared. As a reference with earlier experiments (see Examples 2 and 3) also the pure, non-thickened enzyme solution was incorporated in the test.

To test the efficacy of these various preparations in degrading toxic gluten epitopes, an in vitro digestion test was carried out in which the disappearance of a number of epitopes was followed in time. To that end per slice (approx 10 grams, containing 1.4 g of protein) of a commercially available, toasted white wheat bread (Bolletje, “Engelse toast”), the enzyme in its various presentation forms was applied. So one slice was covered with the raspberry, enzyme containing topping, one slice was covered with the placebo topping (no enzyme activity present) and to one slice the pure, liquid enzyme was added. With both the enzyme containing topping and the pure liquid enzyme, the enzyme activity dosed equaled 20 PPU/g protein present. Five minutes after applying the various products on the toasts, the toasts were minced and mixed with 60 ml (for the control and the liquid variant of the enzyme) and 62.3 ml (for the gel formulation) of a pH 5.0 solution mimicking the gastric liquid (NaCl (4.8 g/L), KCl (2.2 g/L), CaCl₂ (0.22 g/L) and NaHCO₃ (1.5 g/L) 2.3 ml of the same solution containing pepsin from porcine stomach (Sigma, P-7012,) in a concentration of 500 KU/L was added to each of the toasts.

A first sample was obtained at 0 minutes (i.e. 5 minutes after addition of the gastric pepsin solution). Then, gradually the pH of each mixture was lowered to mimic a slow acidification of the stomach content. The pH was lowered from pH 5 to pH 4.5 and 15 minutes after the first sample was collected the second sample is taken. In the following 15 minutes the pH is lowered again to pH 4 and at time point 30 minutes from the beginning of the experiment the third sample is collected. The next samples were collected as follows: at t=45 minutes (after lowering the pH to 3.5); at t=60 minutes (after lowering the pH to 3.0); at t=90 minutes (after lowering the pH to 2.5); then, the pH was lowered to 2.0 and additional samples were taken at 120 minutes and finally at 150 minutes. All samples were frozen instantly in liquid nitrogen and stored afterwards at −80° C.

To measure the level of residual gluten epitope levels in the various preparations, the frozen samples were first subjected to a thorough enzyme inactivation procedure. The still frozen samples were heated to 95 degrees c for 30 minutes, then the pH of the sample was raised to 11-12, then lowered to 2 and finally neutralized to pH 6. Then the samples were heated again for 15 minutes at 95 degrees C. after which a 1 ml aliquot was taken and centrifuged for 30 minutes in an Eppendorf centrifuge. The resulting supernatant contained the water soluble fraction and the pellet the non-water soluble fraction. The levels of remaining T cell stimulatory epitopes in the water soluble fraction were quantified using the monoclonal antibody based competion assays according to Spaenij-Dekking et al, (2004) Gut 53, 1267-1273 (see also the Materials & Methods section). The outcome of these competition assays (the average value measured of two independent measurements) is shown in Table 2. Residual levels of T cell stimulatory epitopes in the pellet fraction were visualized after Western blotting (see Example 3). Similar to the data obtained for the water soluble fraction, also the results of the latter experiment (photographs not shown) confirm the effective breakdown of the various T cell stimulatory epitopes by both enzyme containing preparations.

Collectively, the data obtained clearly indicate that the two enzyme containing preparations, be it in the form of the pure, free enzyme or in the form of a gellified, jam-like product with a high water activity, effectively destroy most T cell stimulatory gluten epitopes if incubated for a period of about 30 minutes under stomach-like conditions. Only the LMW (Low Molecular Weight) fraction seems to be somewhat resistant to enzymatic degradation in the present (in vitro) experimental setup.

TABLE 2
Levels of residual T cell stimulatory epitopes
(in microgram/ml) in the water soluble fraction

| | Sample at | | | | | |
|---|---|---|---|---|---|---|
| | (minutes) | Alpha 9 | Alpha 20 | Gamma 1 | LMW | HMW |
| Enzyme | 0 | 258 | 243 | 212 | 1558 | 158 |
| Placebo | 15 | 94 | 95 | 91 | >2000 | 306 |
| | 30 | 667 | 413 | 625 | >2000 | 333 |
| | 45 | 477 | 531 | 619 | >2000 | 576 |
| | 60 | 318 | 556 | 434 | >2000 | 779 |
| | 90 | 758 | 335 | 567 | >2000 | 994 |
| | 120 | 144 | 539 | 381 | >2000 | 592 |
| | 150 | 120 | 259 | 300 | >2000 | 233 |
| Pure | 0 | | | | | |
| Enzyme | 15 | 46 | 40 | 74 | 568 | 97 |
| Liquid | 30 | 120 | 57 | 112 | >2000 | 144 |
| | 45 | 28 | 21 | 70 | 2010 | 91 |
| | 60 | 27 | 17 | 69 | 668 | 15 |
| | 90 | 41 | 27 | 91 | >2000 | 216 |
| | 120 | 25 | 19 | 66 | 1043 | 152 |
| | 150 | 59 | 31 | 79 | 1310 | 72 |
| Enzyme | 0 | 61 | 35 | 57 | 416 | 99 |
| Topping | 15 | 244 | 103 | 498 | >2000 | 170 |
| | 30 | 136 | 66 | 193 | >2000 | 105 |
| | 45 | 121 | 58 | 121 | >2000 | 160 |
| | 60 | 222 | 95 | 375 | >2000 | 244 |
| | 90 | 73 | 34 | 56 | >2000 | 151 |
| | 120 | 75 | 39 | 104 | 1549 | 132 |
| | 150 | 36 | 21 | 49 | 912 | 83 |

1. A pasteurized food product having a water activity of at least 0.80, preferably at least 0.85 and comprising a proline specific protease.

2. A food product having a water activity of at least 0.80 preferably at least 0.85, and comprising a proline specific protease, whereby the food product comprises less than 1 w/w % of protein or peptides.

3. A food product according to claim 1, which is a gluten-free food product.

4. A food product according to claim 1, which is a condiment, topping, sandwich filling, sauce, beverage or an emulsion such as a spread.

5. A food product according to claim 1, which is a low fat spread.

6. A food product according to claim 1 wherein the proline specific protease has an optimal activity at a pH value between 1 and 7, preferably at a pH value between 2 and 6.

7. A food product according to claim 1, wherein said proline specific protease is derived from Aspergillus or belongs to the S28 family of serine proteases.

8. A process for the preparation of a food product according to claim 1, wherein a proline specific protease is added to said food product followed by subjecting the food product to pasteurisation.

9. A process for the preparation of a food product according to claim 1, wherein a proline specific protease is added to a pasteurized food product.

10. A process according to claim 8 wherein sterile proline specific protease is added to a pasteurized food product.

11. Use of a proline specific protease in the manufacture of a pasteurized food product according to claim 1.

12. Use of a proline specific protease according to claim 11 to prevent any detrimental effect caused by proline-rich food.

13. Use of a proline specific protease according to claim 11 for preventing the clinical symptoms of celiac disease or disorders related therewith.

14. Use according to claim 11, wherein said food product is intended for consumption in combination with a gluten-containing food.

15. Use according to claim 12, wherein said detrimental effect or disorder is related with celiac disease comprise autoimmune disorders, especially type I diabetes, dermatitis herpetiformis, intestinal cancers, intestinal non-Hodgkin's lymphomas, irritable bowel syndrome, autoimmune thyroiditis, collagen diseases, autoimmune alopecia and autoimmune hepatitis, and psychiatric disorders including autism, schizophrenia, ADHD, bipolar mood disorders and depression.

16. Sterile proline specific protease.
